(19) United States
(12) Patent Application Publication
(10) Pub. No.: US 2010/0003707 A1
(43) Pub. Date: Jan. 7, 2010

(54) GLAUCOMA BIOMARKER

(76) Inventor: Alireza Ghaffariyeh, Shiraz (IR)

Correspondence Address: MAHMOUD RAZZAGHI, 865 ESPERANZA PL., CHULA VISTA, CA, 91914, US

(21) Appl. No.: 12/497,626

(22) Filed: Jul. 3, 2009

Related U.S. Application Data
(60) Provisional application No. 61/078,384, filed on Jul. 5, 2008.

(51) Int. Cl. G01N 33/53; G01N 33/68; C12Q 1/02

(52) U.S. Cl. 435/7.92; 436/86; 435/29

(57) ABSTRACT

According the present invention, Brain Derived Neurotrophic Factor (BDNF) in the tears or blood is used as a biomarker for early detection and assessing the progression of POAG. A reduced level of BDNF compared to normal range can be the sign of POAG. Lower levels of BDNF represent more advanced cases of POAG. The invention includes both the method and the analytic kit for performing the method.

This invention has the filing priority of provisional patent application 61/078,384, filed 5 Jul. 2008.

### FIELD OF THE INVENTION

This invention relates to diagnosis, screening and monitoring of patients inflicted with Primary Open Angle Glaucoma (POAG).

### BACKGROUND OF THE INVENTION

Glaucoma is the most widespread neurodegenerative disease of the optic nerve defined by a typical appearance of the optic nerve head and visual field defects. The most common type of glaucoma is POAG. Glaucoma is the second most common cause of visual loss in the world. According to numerous studies, the rate of undiagnosed cases of glaucoma is more than 50%. This high pervasiveness of undiagnosed glaucoma cases stems from POAG developing slowly and asymptomatically until advanced and detectable retinal nerve fiber damages and visual field defects are developed. It is also known to be caused by lack of universal cost-effective POAG screening protocol.

### OBJECTS OF THE INVENTION

Therefore, the object of the invention is to provide a biomarker for early detection of POAG and assessing its progression in the affected cases.

### DESCRIPTION OF THE INVENTION

According the present invention, Brain Derived Neurotrophic Factor (BDNF) in the tears or blood is used as a biomarker for early detection and assessing the progression of POAG. A reduced level of BDNF compared to normal range can be the sign of POAG. Lower levels of BDNF represent more advanced cases of POAG. The invention includes both the method and the analytic kit for performing the method.

Although several different etiologies of POAG have been presented by scientists, it is currently thought they ultimately affect the visual acuity through apoptosis of retinal ganglion cells (RGCs). The RGCs receiving proper chemical signaling from brain neurons and lateral geniculate nucleus (LGN) do not commence the apoptosis. The related brain neurons and LGN impose their neuro-protective effects on the RGCs through a retrograde axonal transport of Neurotrophic factors (NTs), especially the brain-derived neurotrophic factor (BDNF).

BDNF is one of the polypeptide growth factors known to be vital components for building up and preserving of neurons. BDNF is transported to the retinal ganglion cell bodies through a retrograde axonal transportation system and the synaptic connections within. Moreover, BDNF crosses the Blood Brain Barrier (BBB). As a result, the level of this factor in the blood can relatively reflect its concentration in the brain. After crossing the BBB, BDNF are acquired and carried by the platelets, thus serum BDNF is obtained from degranulation of platelets. Therefore, the blood circulation is another possible transport system carrying BDNF to the retinal cells. However, its capacity is significantly less than the axonal BDNF transport system.

BDNF has its own specific receptors called TrkB which exists in all retina layers except in the photoreceptors as well as optic nerve. Experimental research studies suggest that any interruption in the BDNF synthesis or retrograde transport, as occurs when the Internal Ocular Pressure (IOP) is high, can lead to RGCs death, hence glaucoma. This can also explain the significance of the high IOP as the main risk factor of POAG and also the therapeutic effects of lowering the IOP on glaucoma leading to improvement of the BDNF axonal transportation.

Various investigations have shown a serum BDNF decrease in some neurological disorders, like Alzheimer's disease (AD) and Normal Pressure Hydrocephalus (NPH) as well as Multiple Sclerosis (MS). The neurotrophic factors, especially BDNF, can also be produced and released by Muller cells. Consequently, damages or defects affecting these cells can possibly lead to glaucomatous retinal cells death without revealing a significant serum BDNF diminution. Therefore, blood BDNF contents could not exactly reflect the level of this factor in the ocular structures. In such cases, in-tear BDNF level is a better measure for detecting POAG and weighing up its progression.

The levels of neurotrophins (one of which is BDNF) in the tears have been detected and measured in both normal subjects and some abnormal conditions. There are also convincing evidence disclosing the direct connection between the level of some of the growth factors like Hepatocyte Growth Factor (also known as Scatter Factor)(HGF/SF) and Transforming Growth Factor(TGF) with their intraocular levels. Furthermore, in patients with Pseudoexfoliation syndrome (PEX) the increased levels of collagen type IX in the tears correlates with the intraocular amounts of this element which could be used as a useful biochemical marker for the diagnosis of PEX.

Therefore, the level of BDNF in the tears could more clearly reflect the intraocular BDNF content, which is more important in the early diagnosis of POAG and the assessment of its progression. Besides its accuracy and efficacy, detecting and measuring in-tear BDNF level, in general, could be an easy to perform, cost-effective and time-efficient POAG diagnostic method.

### Supporting Study

To support the idea behind the present invention, a study was performed to measure BDNF in the blood and tears of normal subjects and patients with different stages of glaucoma. Afterwards, we compared the results.

### Methods

Twenty three glaucoma patients (41 eyes) as the case group and 13 normal persons as the control group were tested. The control group comprised 3 men and 10 women, with the mean age of 46.1+/−2.2, and without any apparent ocular disease. The case group comprised 15 men and 8 women, ranging in age from 35 to 74 who were assessed by routinely performed clinical and para-clinical investigations. Additionally, in order to determine the age-matched BDNF variations, 8 normal subjects, ranging in age from 24 to 32, who did not have any evident eye disorders were tested. Subjects in all these groups had normal intraocular pressure (IOP).

Based on the clinical findings, the eyes of the patients in the case group were categorized into three sub-groups: 20 eyes with early stages of glaucoma, 18 eyes with moderate to advanced glaucoma and 3 eyes with end-stage glaucoma. In 5 patients, the signs of early stages of glaucoma were found in only one of their eyes.

In addition to the traditional methods of ophthalmologic examinations to confirm the diagnosis of POAG, computerized perimetry by means of Humphrey perimeter (Carl Ziess, Meditec, Germany) with the Swedish interactive threshold algorithms (SITA)-standard 30-2 program and Optical Coherence Tomography (OCT) using Stratus GRL 3000 were conducted.

Samples were taken from all the patients and normal subjects. Peripheral venous blood and tear samples were collected between 9 and 10 am to minimize the possible effects of circadian rhythms on BDNF concentrations. After centrifugation at room temperature (3,500 g for 10 min) the samples were stored at −80° C. until processing.

BDNF levels in blood and tears were determined by ELISA using monoclonal antibodies specific for BDNF (R&D system). The results were subjected to statistical analysis. SPSS software (Version 13 Chicago, Ill., USA) was used for data analysis. Paired T test was used to compare the results. The level of statistical significance was set at P<0.05.

Foveal photosensitivity, mean deviation (MD) of photosensitivity as well as pattern standard deviation (PSD) were looked at in the process of statistical analysis of the data.

BDNF in the blood and tears of all of the subjects were measured. A comparative study was performed once between the case and control groups, and another time among the subjects in the case group in order to find out the importance of this factor in early detection of glaucoma as well as the following up of glaucoma patients.

### Results

Measuring serum BDNF in normal subjects revealed that the amount of this factor in blood decreases with aging. Mean serum BDNF in the subjects, less than 35 years old, was 95.5+/−10.7 ng/ml, while in the group of cases, aged more than 35 this figure changed and dropped to 84.8+/−10.7 ng/ml. However, the results showed that this amount remains almost unchanged after 45.

The results also revealed that almost the same rule applies to BDNF detected in the tears. The mean level of BDNF detected in the tears of the subjects aged less than 35 was 84.3+/−9 ng/ml, while this amount decreased to 77.2+/−9.2 ng/ml in those more than 35 and in the cases aged more than 45, was almost steady.

The average of BDNF levels in the blood was 50.7+/−3 ng/ml in the subjects with the early stage to advanced, and 45.3+/−2 ng/ml in the subjects with the end-stage glaucoma. The BDNF levels in the tears of these two groups were 24.2+/−2.7 ng/ml and 16.5+/−3.1 ng/ml respectively.

There was no significant difference in serum and in-tear BDNF levels according to the subjects' gender (P>0.05).

### Discussion

According to the obtained results, the level of BDNF in the blood and tear remains steady in the normal subjects aged over 45 years. As a result, detection of any change in the level of BDNF in the blood or tear after this age is considered as a pathologic sign. Considering the age, detecting a decreased level of serum or tear BDNF could be a warning for the presence of POAG.

On the other hand, based on the results, there is a negative correlation between the levels of BDNF in the blood and tear of patients with POAG and the severity of the disease. The severity of glaucoma can also be determined by several different factors; such as, the thickness of RNFL, cup to disc ratio, visual field defects and optic disc abnormalities.

It was also noticed that the diminution of BDNF level in the tear is more significant than in the blood. Therefore, measuring the level of BDNF in the tear could be a better indicator not only in early detection of POAG but also in assessing the development and progression of the diseases in affected individuals.

I claim:

1. A method using Brain Derived Neurotrophic Factor (BDNF) in the tears or blood as a biomarker for detection, screening or assessing the progression of glaucoma, the level of BDNF in blood or tears is measured and compared to the normal range, a reduced level of BDNF compared to the normal range indicates the possible presence of glaucoma, lower levels of BDNF represent more advanced cases of glaucoma.

2. An analytic assay or kit for measuring the level of Brain Derived Neurotrophic Factor (BDNF) in the tears or blood for detection, screening or assessing the progression of glaucoma, the measured level of BDNF in the tears or blood is compared to the normal range, a reduced level of BDNF compared to the normal range indicates the possible presence of glaucoma, lower levels of BDNF represent more advanced cases of glaucoma.

3. The invention of claim 2 wherein the analytic assay or kit is ELYSA or ELA.
